(19) United States
(12) Patent Application Publication
(10) Pub. No.: US 2006/0058739 A1
(43) Pub. Date: Mar. 16, 2006

(54) Self-occluding sheath

(76) Inventors: Steven Smith, Chicago, IL (US); Robert L. Vogelzang, Chicago, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/200,927

(22) Filed: Aug. 10, 2005

Related U.S. Application Data
(60) Provisional application No. 60/600,586, filed on Aug. 11, 2004.

(51) Int. Cl. A61M 5/00

(52) U.S. Cl. 604/246

(57) ABSTRACT

A sheath has a cannulated portion having a wall with an inner diameter and an outer diameter. A valve is formed within wall. The valve has a first position occluding fluid flow through the sheath and a second position permitting fluid flow through the sheath.

### TECHNICAL FIELD

This patent relates to sheaths used for the introduction of catheters into blood vessels, and more particularly, this patent relates to self-occluding sheaths useful in the placement of large-bore dialysis catheters, and the like.

### BACKGROUND

Splitable or peelable sheaths provide ability to easily and reliably place soft, tunneled catheters, such as dialysis catheters, into blood vessels. The catheter itself is usually made of a soft, flexible material that prohibits direct insertion into the blood vessel or insertion over a guide wire. The splitable sheath allows the catheter to be inserted into the blood vessel from within the sheath. Then, once in position within the blood vessel, the splitable sheath is separable into two or more sections allowing withdrawal from the blood vessel while leaving the catheter behind.

The commonly practiced procedure for placing a soft catheter within a blood vessel is to first enter the vessel using a cannula and to insert a guide wire into the vessel via the cannula. After withdrawing the cannula, a dilator is inserted into the vessel over the guide wire. The splitable sheath is disposed over the dilator as it is guided into the vessel. The sheath is opened by extracting the dilator allowing the catheter to be inserted into the blood vessel via the sheath. The sheath is then withdrawn while simultaneously being separated into usually two pieces leaving the catheter in place within the vessel.

With the dilator extracted, the sheath is patent, allowing fluids, such as blood to flow from vessel. A more serious complication, however, is the possibility of air embolism. During the period following extraction of the dilator and prior to insertion of the catheter, it is possible for the patient to inhale, or even gasp, and create a sudden negative intra-thoracic pressure. As a result, air may be aspirated into the vessel via the patent sheath potentially leading to massive, fatal air embolism. Physicians during these procedures will often instruct the patient not to inhale, to slowly exhale or even to hum during this portion of the procedure to reduce the possibility of the patient inhaling. And, while the potential for the occurrence of this complication has existed for years, the increasing diameter of modern dialysis catheters, partly made possible as a result of the splitable sheaths, increases the risk of complication.

Being able to pinch or clamp the sheath shut after withdrawing the dilator is sometimes effective to occlude the sheath and prevent the inrush of air. Unfortunately, the patient's size may require insertion of the sheath up to its entire length, e.g., tall or obese patients. Also, the sheath is not made to be pinched, and in fact may be made to resist crushing to ensure patency. Furthermore, pinching and more particularly clamping may damage the sheath leaving it partially occluded preventing insertion of the catheter. The surface of the sheath may also be slick with blood or other fluids, making grasping and pinching difficult. And, having to grasp and pinch the sheath restricts at least one of the physician's hands until the catheter is at least partially inserted therein.

Therefore, there is a need for a sheath suitable for use in the placement of catheters, such as large bore, soft dialysis catheters.

### BRIEF DESCRIPTION OF THE DRAWINGS

FIG. 1 is a front view of a sheath in accordance with one of the herein described embodiments;

FIG. 2 is a cross-section view taken, along line 2-2 of FIG. 1:

FIG. 3 is a cross-section view taken along line 3-3 of FIG. 1

FIG. 4 is similar to the view of FIG. 3 with the sheath in a second configuration;

FIG. 5 is a front view of the sheath in a second configuration;

FIG. 6 is a partial view of a sheath in accordance with another of the described embodiments;

FIG. 7 is a partial view of the sheath shown in FIG. 6 in a second configuration;

FIG. 8 is a cross-section view taken along line 8-8 of FIG. 7;

FIG. 9 is a cross-section view of a valve;

FIG. 10 is a cross-section view of an alternative valve structure; and

FIG. 11 is a further cross-section view the valve structure illustrated in FIG. 10.

FIG. 12 is a cross-section view of a sheath in accordance with another of the herein described embodiments in an open state.

FIG. 13 is a cross-section view of the sheath illustrated in FIG. 13 in an occluded state.

FIG. 14 is a cross-section view of a portion of a sheath in accordance with another of the herein described embodiments in an open state.

FIG. 15 is a partial cross-section view of a portion of a sheath in accordance with another of the herein described embodiments in an open state.

### DETAILED DESCRIPTION

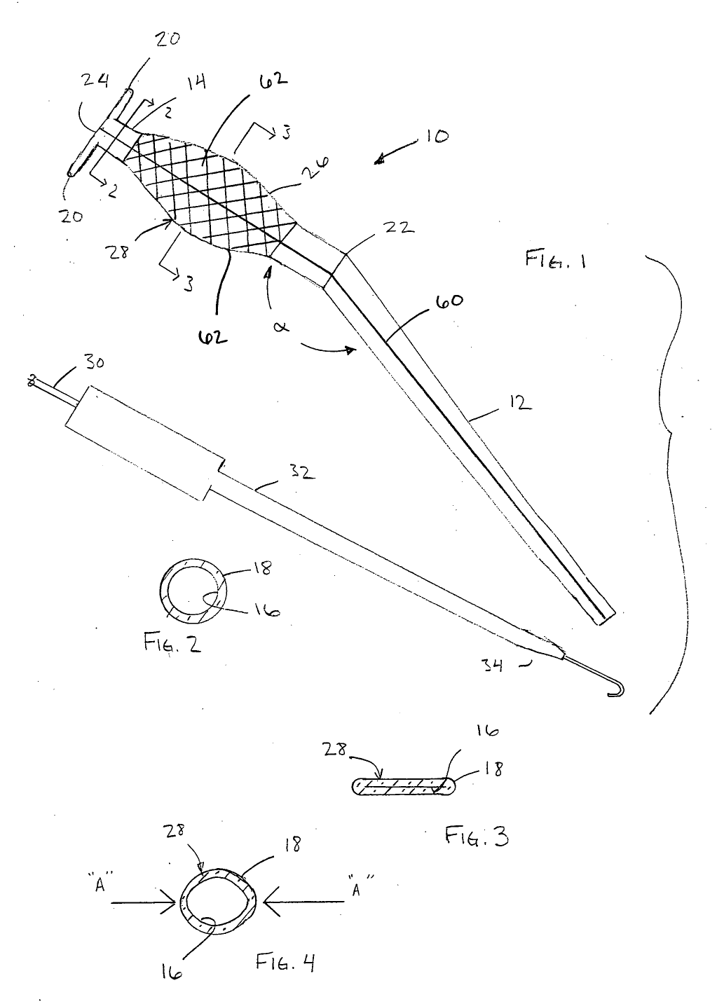

FIG. 1 illustrates a sheath 10 having a first cannulated portion 12 and a second cannulated portion 14 formed at an angle α to the first portion 12. Both the first and second portions have an inner diameter 16 and an outer diameter 18 (FIG. 2); the inner diameter 16 being selected to correspond to a catheter to be placed within a vessel by use of the sheath 10. Typically the sheath 10 may have an inner diameter 16 from about 8 French (F) to about 16 F (2.66 mm to about 5.33 mm). In practice, the inner diameter 16 may be selected such that the catheter will substantially entirely occupy the inner diameter 16 of the sheath while allowing the catheter to be easily pushed through the length of the sheath 10. The sheath need not be circular in cross-section, but may be oval or otherwise shaped. The first portion 12 may be made of a compliant, compressible material to facilitate occluding the sheath and sealing the cannula. For example, the first portion may be a natural or synthetic rubber, silicone, latex or other suitable material. The second portion 14 may be a Teflon® other flexible, but sufficiently rigid material to allow the sheath 10 to be guided over a dialator into a patient.

The sheath 10 may be a peelable or splitable sheath. That is, once the catheter is placed within the vessel, it is possible to separate the sheath 10 into two or more portions so that it may be removed from around the catheter and from the patient. The sheath 10 may therefore include tabs, such as tabs 20 that may be grasped and pulled outwardly to separate the sheath 10. The sheath 10 may further be formed with one or more score lines 60 along its length to facilitate such separation.

The portion 12 may have a length sized to permit operable insertion of the sheath 10 up to the joint 22 between the first portion 12 and the second portion 14. Much in the same way the sheath 10 may be constructed in a variety of different diameters, the length of the first portion 12 may be varied for use with patients of different ages, heights and weights. The angle α may be in the range from about 15 degrees to about 45 degrees and more frequently be between about 20 degrees to about 30 degrees and facilitates positioning of the second portion 14, and hence the insertion end 24 of the sheath 10, toward the physician during use of the sheath. Alternatively, the angle α may be such that the first portion 12 may be aligned to extend upwardly even to vertical. Such an arrangement permits introducing a column of sterilized water or saline solution within the portion 12. The column of water or saline acting effectively as a valve to prevent aspiration of air.

An outer surface region 26 of the second portion 14 may have a non-slip surface treatment. As shown in the figures, the region 26 may have a knurled or textured surface treatment. Alternatively, the region 26 may have a non-slip coating or appliqué. The region 26 is provided to facilitate grasping of the sheath by the physician during use. Frequently the sheath 10 will have blood or other fluids coating its outer surface making the otherwise smooth outer surface of the sheath slippery and difficult to grasp. The region 26, whether provided by texturing, coating or appliqué, enhances the ability of the physician to grasp the sheath 10 during use.

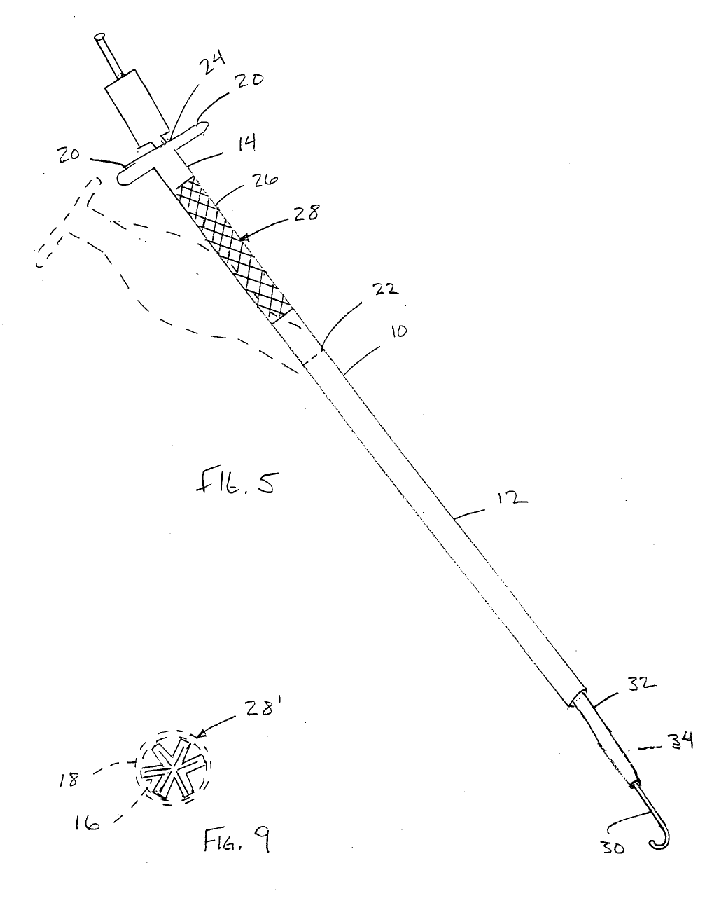

The sheath 10 may further be formed, generally in the region 26 but not necessarily so, with an integrally formed valve 28. In fact, the valve 28 may be formed anywhere along the length of the sheath 10 accessible to the physician during a catheter placement or other procedure for which the sheath may be used. With continued reference to FIG. 1 and additional reference to FIGS. 3 and 4, the valve 28 may be an in situ formed flattened portion of the sheath 10. As shown in FIG. 3, absent application of external forces, the valve 28 is normally closed substantially occluding the sheath 10 within the second portion 14. Application of force, such as a pinching force indicated by the arrows “A” in FIG. 4, causes an elastic deformation and opening of the valve 28. To facilitate operation of the valve 28, the sheath 10 entirely, the second portion 14 or the immediate region incorporating valve 28 may be formed using a plastic material having shape memory characteristics. Such materials are well known to the person of ordinary skill in the art and need not be listed here. However, many plastic materials typically used for medical devices or appliances will have sufficient elasticity resulting in the desired operation of the valve 28. As shown in FIG. 3, the valve 28 is normally closed but is openable upon application of a pinching or similar force to an outer wall of the sheath 10 in the vicinity of the valve 28, as shown in FIG. 4.

Referring to FIG. 5, the sheath 10 is positioned within a vessel using well known techniques. First a cannula (not depicted) is used to pierce the vessel and a guide wire 30 is inserted through the cannula into the vessel. The cannula is removed, and a dilator 32 with the sheath 10 disposed thereon is guided over the guide wire 30 into the vessel. As shown the sheath 10 is sufficiently flexible such that the angle α is removed from the sheath 10 with the dilator 32 in place. Moreover, the dilator 32 being inserted through the sheath 10 holds the valve 28 open.

With the sheath 10 positioned within the vessel, the guide wire 30 and the dilator 32 are withdrawn. The valve 28 closes behind the dilator 32 as it is withdrawn past the valve 28 occluding the sheath 10. Removal of the dilator also allows the first portion 12 and the second portion 14 to return to their angled orientation.

Referring again to FIG. 4, with pressure applied to the valve 28, the inner diameter 16 of the sheath 10 in the vicinity of the valve 28 is not perfectly circular, and may by oval. The dilator 32 may therefore be formed with a substantially oval or shaped tip 34 to enhance the closure of the sheath 10 as the dilator 32 is withdrawn past the valve 28.

With the sheath 10 in place and the dilator 32 removed, the valve 28, which is normally closed, substantially inhibits the aspiration of air into the vessel, for example, as a result of the patient inhaling. Therefore, the physician need not grasp the sheath pinching it closed and may have both hands free to ready the catheter, to align it with the insertion end 24 of the sheath 10 and to begin insertion of the catheter into the sheath 10. Once the catheter reaches the valve 28, the soft catheter material generally will not be sufficiently rigid to push the valve 28 open, although there may exist procedures for the placement of sufficiently rigid catheters or other devices where such is the case. With the catheter occupying the portion of sheath from the insertion end 24 to the valve 28, the sheath 10 is substantially occluded by both the valve 28 and the catheter, and the risk of air aspiration is minimal. The valve 28 may be opened by applying a pinching force to the sheath 10 as shown in FIG. 4. The valve 28 therefore opens allowing the catheter to be pushed passed the valve 28, along the length of the catheter and into the desired vessel. The pressure on the valve 28 may then be released. Whether or not the valve 28 closes completely as a result of the presence of the catheter within the sheath 10 is not detrimental to the operation of the sheath 10, as the catheter itself, occupying the sheath 10, will substantially occlude the sheath 10. The sheath 10 may then be separated and removed from the patient leaving the catheter in place, as is well know. The score lines 60 formed with in the wall of the sheath 10 to facilitate splitting of the sheath 10 may also extend along either side of the valve 28. Thus, the valve 28 being formed within a wall of the sheath 10 also separates along the score lines formed within the sheath 10 when the sheath 10 is split for removal. The score lines 60 may be disposed at virtually any location about the circumference of the sheath 10, and, while depicted extending through a surface 62 of the valve 28, the score lines may be arranged to extend along an edge 64 of the valve 28 formed in the sheath 10.

In an alternative embodiment the valve 28 may be formed to be normally open. In such an embodiment, the material of the sheath 10, the second portion 14 or immediately adjacent the valve 28 may be made sufficiently elastic to allow it to be easily squeezed, such as by applying a pinching force, to allow it to occlude the sheath 10. The region 26 may still be provided to facilitate the physician gripping and squeezing the sheath 10 to close the sheath 10 against air aspiration. The remaining regions of the sheath 10 may be of the same material or may be a different material designed to resist collapsing under squeezing pressure to ensure sheath patency.

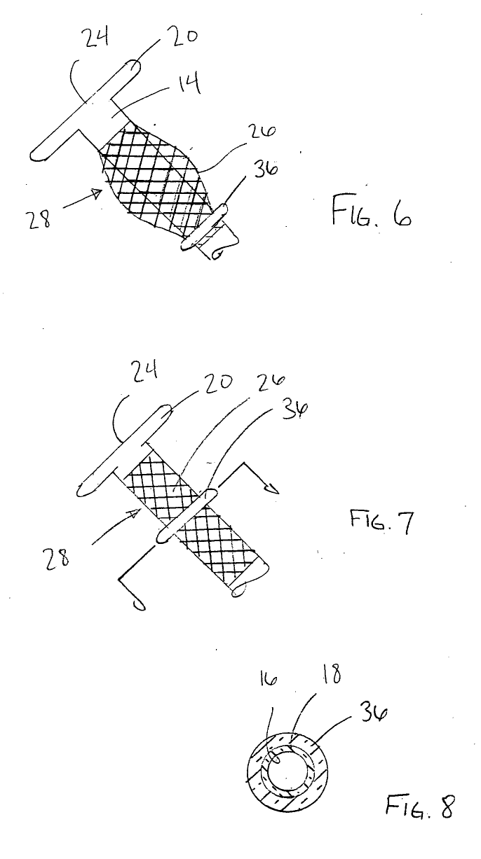

In still another embodiment, illustrated in FIGS. 6-8, a ring 36 may be provided disposed about the sheath 10 adjacent the second portion 14. The ring has an outer diameter 42 and an inner diameter 44. The ring 36 is slideable along the sheath 10 over the region 26 to be positioned adjacent the valve 28. In this manner, the ring 36 may be used to lock the valve 28 open. The ring 36 may be substantially rigid, and thus slideable along the sheath 10, or it may be elastic and rollable along the sheath 10. The ring 36 need not be circular in cross-section and may have a sleeve or sleeve-like construction and may further include a flattened or contoured that allows the user to position a thumb or finger to facilitate movement of the ring along the sheath 10. It is likely the ring 36 will also be removed when the sheath 10 is split and removed. The ring 36 may be made of a sufficiently brittle material that compressing its outer diameter causes it to crack and split allowing it to be removed. The ring 36 may also be formed to include one or more failure point, scores, notches or the like, that allow the ring 36 to be easily fractured and removed from the sheath 10.

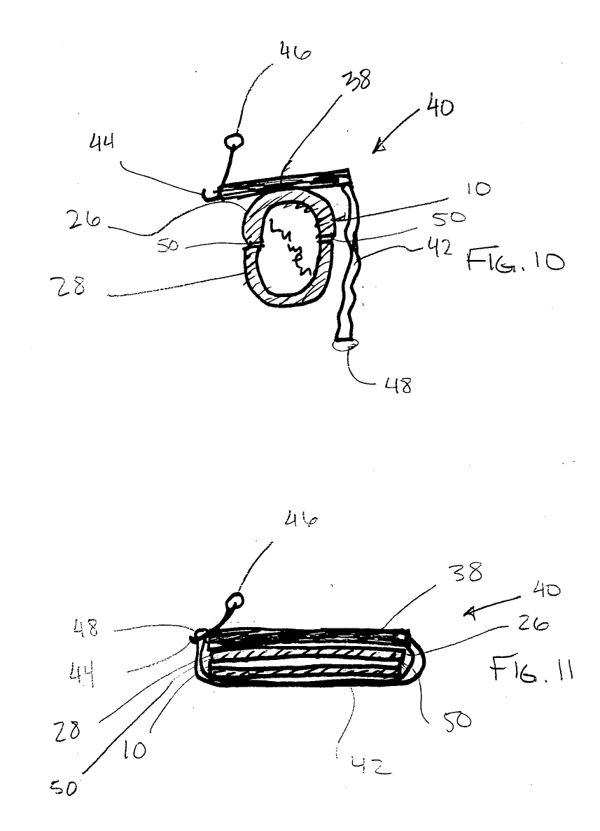

In still another alternative embodiment illustrated in FIGS. 10 and 11, an integral clamp 40 may be secured to the sheath 10 adjacent the valve 28. The claim 40 may include a bar 38 that is bonded, welded or otherwise formed with or attached to the outer surface region 26 of the sheath 10, An elastic band 42 may be wrapped around the sheath 10 and secured by a retainer 44 including a release arm 46 (FIG. 11). For example, the elastic band 42 may be formed with a knob or bulb 48 at one end that is received through an aperture formed in the retainer 44. Rotation of the release arm 46 allows the bulb to be released from the aperture to allow the elastic band 42 to become loose. This, in turn, allows the valve 28 to open to a substantially circular cross-section for passage of the catheter (FIG. 10). The retainer may alternatively have a hook portion that is received through an aperture formed in the band 42. The bar 38 is secured to one side of the sheath 10 so that it does not interfere with the peel or separablity of the sheath 10 along separation lines 50.

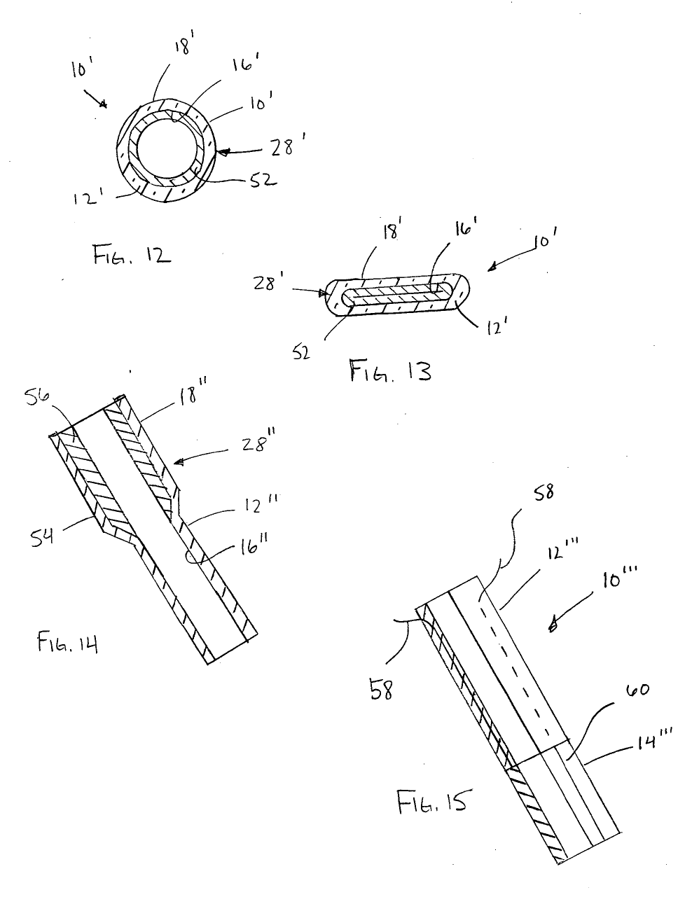

FIGS. 12 and 13 illustrate a sheath 10′ wherein the valve 28′ may include a coating 52 on the inner diameter 16′ of the first portion 12′. The coating 52 may be a compressible material or a material that enhances the sealing properties of the valve 28′, especially the inner diameter 16′. For example, the coating 52 may be a layer of natural or synthetic rubber, latex, silicone or other suitable polymer material that generally seals against itself and may be compressible to enhance sealing.

FIG. 14 illustrates a sheath 10″ wherein the valve 28″ may be an enlarged portion 54 of the first portion 12′. That is, the inner diameter 16″ in the area of the valve 28″ is larger than the inner diameter 16″ in the area adjacent the valve 28″. Within the larger diameter portion, there may be provided a sealing material 56, such as natural or synthetic rubber, latex, silicone, or other suitable polymer material.

FIG. 15 illustrates a sheath 10′″. As noted, the first portion 12′″ may be a different material than the second portion 14′″. For example, the first portion 12′″ may be an easily compressible and sealable material incorporating the valve 28′″. The second portion 14′″ may be Teflon® material or other similar material that is relatively rigid yet sufficiently flexible to allow placement of the sheath 10′″. To facilitate splitting the sheath 10′″ a “ripper” 58 is disposed within the wall 60 of the first portion 12′″. The rippers 58 may be Teflon® strands, nitonal wires, monofilament or other wire-like elements. The rippers 58′″ may extend the entire length of the sheath 10′″. Alternatively, the rippers 58 may be disposed only in the first portion 12′″. The rippers 58 may be aligned with scores lines 60 formed in the second portion 14′″ so that as the first portion 12′″ is split by the rippers 58, the sheath 10′″ continues to split through the second portion 14′″ along the score lines 60.

The foregoing described embodiments relate to a sheath including a valve formed in a cannulated wall portion of the sheath that is either normally closed and openable or normally open and closable by application of opposing force to the wall of the sheath in the vicinity of the valve. The valve acts to occlude the sheath so as to prevent fluids from being either aspirated or expelled via the sheath. The valve 28 need not close along a single line of contact as shown in FIG. 4, and it may close in an accordion-like manner along multiple lines of contact, such as illustrated for the valve 28′ in FIG. 9. Other structures providing valve-like closing action that may be incorporated within the cannulated wall of the sheath may also be used and are contemplated as being part of the instant invention.

The invention has also been described in terms of several preferred embodiments for the placement of a catheter. It is readily understood that a sheath in accordance with the teachings of the instant invention will have numerous applications, particularly as they relate to procedures involving entry of a vessel, such as a blood vessel.

A kit may include a sheath in accordance with any of the herein described embodiments. For example, the sheath may have a cannulated portion with a wall and a valve formed within wall. The valve may have a first position occluding fluid flow through the sheath and a second position permitting fluid flow through the sheath. The kit may further include a dialator and guide wire used in connection with placing the sheath. The dilator includes a tip. The tip may have a non-circular, cross-section corresponding to the cross-section of the valve when the valve is in an occluded or closed position. The tip therefore assists and facilitates opening of the valve during placement of the sheath.

While the present disclosure is susceptible to various modifications and alternative forms, certain embodiments are shown by way of example in the drawings and these embodiments will be described in detail herein. It will be understood, however, that this disclosure is not intended to limit the invention to the particular forms described, but to the contrary, the invention is intended to cover all modifications, alternatives, and equivalents falling within the spirit and scope of the invention defined by the appended claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Unless a claim element is defined by reciting the word “means” and a function without the recital of any structure, it is not intended that the scope of any claim element be interpreted based on the application of 35 U.S.C. §112, sixth paragraph.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated-herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

We claim:

1. A sheath comprising:
a cannulated portion having a wall with an inner diameter and an outer diameter; and
a valve formed within the wall, the valve having a first position occluding fluid flow through the sheath and a second position permitting fluid flow through the sheath.

2. The sheath of claim 1, a portion of the outer diameter being a non-slip surface.

3. The sheath of claim 2, wherein the non-slip surface comprises a textured non-slip surface.

4. The sheath of claim 2, wherein the non-slip surface comprises one of a non-slip coating and a non-slip appliqué.

5. The sheath of claim 1, the sheath having a first cannulated portion and a second cannulated portion formed at an angle to the first cannulated portion.

6. The sheath of claim 5, wherein the angle is between about 10 degrees to about 45 degrees.

7. The sheath of claim 1, the sheath being separable longitudinally into a first portion and a second portion for removal of the sheath.

8. The sheath of claim 1, wherein the valve comprises a collapsible portion of the wall.

9. The sheath of claim 8, wherein the wall collapses along a single line of contact.

10. The sheath of claim 8, wherein the wall collapses along multiple lines of contact.

11. The sheath of claim 1, comprising a valve retainer disposed about the sheath, the valve retainer being positionable for retaining the valve in one of the first and second positions.

12. The sheath of claim 11, the valve retainer comprising a ring disposed about the sheath.

13. The sheath of claim 1, comprising a seal enhancing coating disposed on the inner diameter in a portion of the wall including the valve.

14. The sheath of claim 10, comprising a first material forming a first portion of the wall in a first portion of the wall including the valve and a second material forming a second portion of the wall in a second portion of the wall separate from the valve.

15. The sheath of claim 10, the inner diameter being enlarged in a portion of the wall including the valve and a seal enhancing material being disposed within the enlarged inner diameter.

16. The sheath of claim 1, comprising a ripper for facilitating longitudinal separation of the sheath into a first and a second portion.

17. A sheath comprising:
a first cannulated member and a second cannulated member joined to the first cannulated member and forming an angle between the first cannulated member and the second cannulated member;
a valve disposed within a wall portion of the first cannulated member; and
a non-slip surface treatment being applied to an outer surface of the wall in the vicinity of the valve.

18. The sheath of claim 17, the first cannulated member being a first material and the second cannulated member being a second material, the first material being an enhanced sealing material.

19. A kit comprising:
a sheath having a cannulated portion with a wall and a valve formed within wall, the valve having a first position occluding fluid flow through the sheath and a second position permitting fluid flow through the sheath;
dilator; and
a guide wire.

20. The kit of claim 14, wherein the dilator has a tip, the tip having a non-circular cross-section corresponding to a cross-section of the valve when the valve is in the second position.
